(19) United States
(12) Patent Application Publication
(10) Pub. No.: US 2019/0195763 A1
(43) Pub. Date: Jun. 27, 2019

(54) SENSOR ELEMENT AND SENSOR APPARATUS

(71) Applicant: KYOCERA Corporation, Kyoto (JP)

(72) Inventor: Yutaka IKEDA, Ikoma-shi, Nara (JP)

(73) Assignee: KYOCERA Corporation, Kyoto (JP)

(21) Appl. No.: 16/329,071

(22) PCT Filed: Aug. 30, 2017
(86) PCT No.: PCT/JP2017/031122

§ 371 Date: Feb. 27, 2019

(30) Foreign Application Priority Data
Aug. 31, 2016 (JP) 2016-169403

(51) Int. Cl. G01N 5/02; G01N 33/497

(52) U.S. Cl. CPC G01N 5/02; G01N 33/497

(57) ABSTRACT

A sensor element includes a substrate and reactive portions disposed on the substrate and configured to react to a specific component. The reactive portions include a first reactive portion and a second reactive portion having a lower reactivity than the first reactive portion with respect to a component to be detected in a sample. The reactivity of the first reactive portion with respect to the component to be detected is higher than the reactivity with respect to a noise component in the sample other than the component to be detected.

### CROSS-REFERENCE TO RELATED APPLICATION

The present application claims priority to and the benefit of Japanese Patent Application No. 2016-169403 filed Aug. 31, 2016, the entire contents of which are incorporated herein by reference.

### TECHNICAL FIELD

The present disclosure relates to a sensor element and a sensor apparatus.

### BACKGROUND

A known sensor detects and measures a specific component in a fluid. For example, patent literature (PTL) 1 discloses a gas sensor that includes a diaphragm and a plurality of sensitive membranes on the surface of the diaphragm.

### CITATION LIST

### Patent Literature

PTL 1: JP2014153135A

### SUMMARY

A sensor element according to an embodiment of the present disclosure includes a substrate and reactive portions disposed on the substrate and configured to react to a specific component. The reactive portions include a first reactive portion and a second reactive portion having a lower reactivity than the first reactive portion with respect to a component to be detected in a sample. The reactivity of the first reactive portion with respect to the component to be detected is higher than the reactivity of the first reactive portion with respect to a noise component in the sample other than the component to be detected.

A sensor apparatus according to an embodiment of the present disclosure includes a sensor element and a controller. The sensor element includes a substrate and reactive portions disposed on the substrate and configured to react to a specific component. The controller is configured to calculate a value related to a component in a sample on the basis of a signal output from the sensor element in accordance with a reaction of the reactive portions. The reactive portions include a first reactive portion and a second reactive portion having a lower reactivity than the first reactive portion with respect to the component to be detected. The reactivity of the first reactive portion with respect to the component to be detected is higher than the reactivity of the first reactive portion with respect to a noise component in the sample other than the component to be detected.

### BRIEF DESCRIPTION OF THE DRAWINGS

In the accompanying drawings:

FIG. 1 is a schematic perspective view of a sensor element according to an embodiment;

FIG. 2 is a functional block diagram illustrating the schematic configuration of a sensor apparatus including the sensor element in FIG. 1;

FIG. 3A illustrates an example of the measurement principle of the sensor element in FIG. 1;

FIG. 3B illustrates an example of the measurement principle of the sensor element in FIG. 1;

FIG. 4 illustrates simulation results;

FIG. 5 illustrates simulation results;

FIG. 6 illustrates simulation results;

FIG. 7 illustrates simulation results;

FIG. 8 illustrates an example of noise components in a simulation;

FIG. 9 illustrates an example of selectivity;

FIG. 10 illustrates simulation results;

FIG. 11 illustrates simulation results; and

FIG. 12 illustrates simulation results.

### DETAILED DESCRIPTION

An embodiment is described below in detail with reference to the drawings.

<Sensor Element>

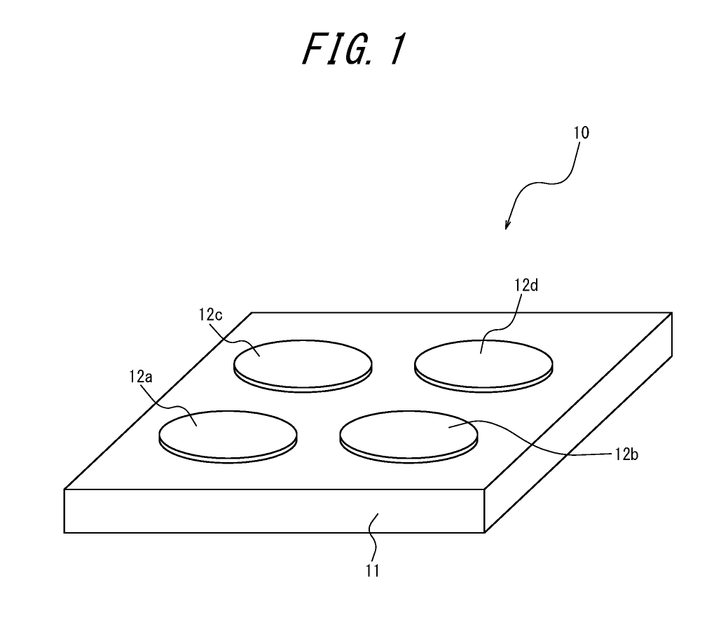

FIG. 1 is a schematic perspective view of a sensor element 10 of the present disclosure.

The sensor element 10 can detect a component that is the target of detection (component to be detected) in a fluid for measurement. The sensor element 10 is provided with a substrate 11, reactive portions 12, and detectors 13. The sensor element 10 illustrated in FIG. 1 includes a first reactive portion 12a, a second reactive portion 12b, a third reactive portion 12c, and a fourth reactive portion 12d, and a first detector 13a, a second detector 13b, a third detector 13c, and a fourth detector 13d.

The number of reactive portions 12 included in the sensor element 10 is not limited to four. It suffices for the sensor element 10 to include two or more reactive portions 12, and for the number of detectors 13 to correspond to the number of reactive portions 12. The plurality of detectors 13 may, for example, be disposed on the substrate 11 in correspondence with the plurality of reactive portions 12. The detectors 13 are not depicted in FIG. 1.

In the present disclosure, the first to fourth reactive portions 12a to 12d are referred to as reactive portions 12 when not distinguishing between the reactive portions. The first to fourth detectors 13a to 13d are referred to as detectors 13 when not distinguishing between the detectors.

The substrate 11 is a deformable member. The substrate 11 may, for example, be a thin substrate functioning as a diaphragm. Specifically, the substrate 11 may be an n-type Si substrate, for example.

The reactive portions 12 can react to specific components. The reactive portions 12 are disposed on the substrate 11. The reactive portions 12 may, for example, be film-shaped members. It suffices for the reactive portions 12 to be made of material that deforms by adsorbing a specific component. The reactive portions 12 may, for example, be made of material such as polystyrene, chloroprene rubber, polymethyl methacrylate, or nitrocellulose.

If each reactive portion 12 is made of a different material, then each reactive portion 12 can be provided with different selectivity with respect to specific components. In other words, the degree of reaction to specific components can be changed, or the reactive portions 12 can be caused to react to different components. Here, selectivity refers to the reactivity (or sensitivity) to each specific component. Specifically, when a plurality of types of components are supplied to one reactive portion 12 at the same concentration, the selectivity is the contribution ratio of each component to deformation of the reactive portion 12.

The detectors 13 can detect that the reactive portions 12 have reacted to a specific component. The detectors 13 are, for example, piezoresistive elements disposed on the substrate 11. The detectors 13 may, for example, form a Wheatstone bridge circuit that contains four piezoresistive elements. The detectors 13 may, for example, be formed by diffusing boron (B) on the substrate 11.

The sensor element 10 can detect a specific component by virtue of having the aforementioned configuration. Specifically, the reactive portions 12 first react to a specific component and deform. The substrate 11 deforms in accordance with deformation of the reactive portions 12. Stress is applied to the detectors 13 by deformation of the substrate 11, changing the electrical resistance of the detectors 13. Consequently, the output of the detectors 13 varies, allowing the sensor element 10 to detect a specific component.

Accordingly, if a component to be detected is included in a fluid for measurement supplied to the sensor element 10, for example, the sensor element 10 can detect the component.

The detectors 13 output an electric signal corresponding to the reaction to a specific component. In the present disclosure, the signal output by the detectors 13 is also referred to as “sensor output”. The sensor output may, for example, be a voltage value.

<Sensor Apparatus>

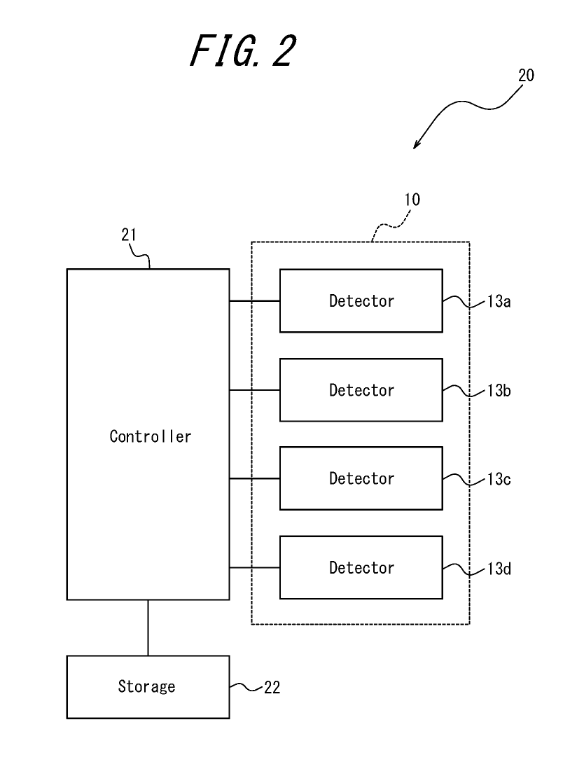

FIG. 2 is a functional block diagram illustrating the schematic configuration of a sensor apparatus 20.

The sensor apparatus 20 in FIG. 2 includes the sensor element 10 of FIG. 1. Specifically, the sensor apparatus 20 includes a controller 21, a storage 22, and the sensor element 10 (detectors 13), as illustrated in FIG. 2. On the basis of the state of reaction to a component in the reactive portions 12, the sensor apparatus 20 can detect a value related to a component included in the fluid for measurement. For example, the sensor apparatus 20 can calculate the concentration of the component to be detected included in the fluid for measurement. The value related to a component included in the fluid for measurement is not limited to the concentration, however, and may be any value, such as an index represented as a numerical value. Furthermore, the value related to a component included in the measured sample is not limited to a value related to the component to be detected and may, for example, be a value related to components other than the component to be detected. In the present disclosure, the sensor apparatus 20 is described below as calculating the concentration of the component to be detected included in the fluid for measurement.

The controller 21 is a processor that controls and manages the sensor apparatus 20 overall, starting with the functional blocks of the sensor apparatus 20. The controller 21 is a processor, such as a central processing unit (CPU), that executes a program prescribing control procedures. Such a program may, for example, be stored in the storage 22, on an external storage medium connected to the sensor apparatus 20, or the like.

The storage 22 may, for example, be a semiconductor memory, a magnetic memory, or the like. The storage 22 can, for example, store various information and/or programs for operating the sensor apparatus 20. The storage 22 may also function as a working memory.

<Principle of Measurement of Component to be Detected>

The principle of measurement of the component to be detected included in the fluid for measurement is now described. Measurement of the concentration of the component to be detected mainly includes a step of calculating the concentration of the component to be detected and a step of generating a mathematical formula for concentration calculation.

In the present embodiment, an example of the controller 21 calculating the concentration of the component to be detected is described. Here, the fluid for measurement is described as being a gas.

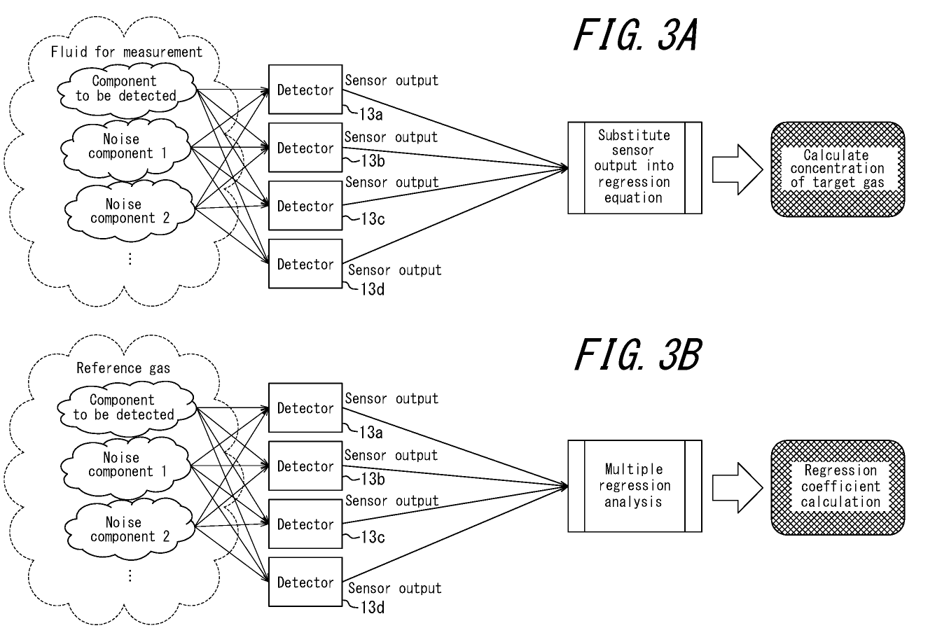

FIG. 3A and FIG. 3B illustrate an example of the measurement principle of the sensor apparatus 20. On the basis of FIG. 3A, calculation of the concentration of the component to be detected in the fluid for measurement is described.

As illustrated in FIG. 3A, the controller 21 performs a mathematical operation by substituting the sensor output of each detector 13 into a predetermined mathematical formula to calculate the concentration of the component to be detected. The predetermined mathematical formula is, for example, obtained as a regression equation for calculating the concentration of the component to be detected using a method such as multiple regression analysis.

FIG. 3B illustrates the calculation of regression coefficients using multiple regression analysis. The calculation method of the regression equation is described with reference to FIG. 3B.

First, to calculate the regression coefficients, a plurality of reference gases are prepared. The plurality of reference gases are components assumed to be included in the fluid for measurement (assumed components). To perform multiple regression analysis, the plurality of reference gases include the assumed components at predetermined concentrations, and the concentrations of the assumed components differ for each reference gas. Next, the plurality of reference gases are supplied to the reactive portions 12 of the sensor element 10. Sensor output corresponding to the selectivity of each reactive portion 12 is then obtained from the detectors 13. As a result, multiple regression analysis can be performed on the basis of the sensor output from the detectors 13 to calculate the regression coefficients. Sufficient types of gases for performing multiple regression analysis are prepared as the plurality of reference gases.

Generation of the regression equation is now described in greater detail using an example in which the sensor element 10 includes the first and second reactive portions 12a and 12b.

The sensor output (Y₁, Y₂) of the first and second detectors 13a, 13b can be represented by Equation 1 below, where the selectivity of the component to be detected is A₁ for the first reactive portion 12a and A₂ for the second reactive portion 12b, the selectivity of the noise component is B₁ for the first reactive portion 12a and B₂ for the second reactive portion 12b, X_{A} is the concentration of the component to be detected, and X_{B} is the concentration of the noise component. In the present disclosure, the noise component is the component included in the fluid for measurement other than the component to be detected. The constant terms (Z₁, Z₂) in Equation 1 are, for example, signals output due to manufacturing error or the like, even when no fluid is being supplied.

Y₁=(A₁×X_{A})+(B₁×X_{B})+Z₁ (constant term)

Y₂=(A₂×X_{A})+(B₂×X_{B})+Z₂ (constant term) Equation 1:

The regression coefficients α, β, and γ in the regression equation below (Equation 2) are calculated by performing multiple regression analysis using the sensor output (Y₁, Y₂) of the first and second detectors 13a, 13b obtained with Equation 1 for the plurality of reference gases and the concentration (X_{A}) of the component to be detected.

X_{A}=α×Y₁+β×Y₂+γ Equation 2:

When the selectivity of the reactive portions 12 (A₁, A₂, B₁, B₂) is known, the multiple regression analysis may be performed by a computer simulation instead of measuring reference gases. The selectivity can be determined by preparing a gas composed only of one assumed component (single gas) for each of the assumed components and comparing the sensor output for each single gas.

The regression equation (Equation 2) can be generated in this way. The sensor apparatus 20 (controller 21) can calculate the concentration of the component to be detected by mathematical processing to substitute the sensor output of each detector 13 when the fluid for measurement is supplied to the reactive portions 12 into Y₁ and Y₂ of the regression equation (Equation 2).

For the sake of explanation, Equations 1 and 2 are simplified above. Equations 1 and 2 that are adjusted to the measurement conditions and the like may be used in an actual sensor apparatus 20. For example, the equations above include one term for the noise component, but a separate term may be set for each noise component. Only Y₁ and Y₂ have been set, since two reactive portions 12 are provided in the above explanation, but terms Yₘ (m=1, 2, . . . , n) may be set in accordance with the number (n) of reactive portions 12.

When the sensor apparatus 20 is used, it is assumed that the measurement results will shift from the true values depending on how the fluid for measurement is supplied. For example, due to the effect of the measurement atmosphere, the component to be detected may be supplied to the sensor element 10 at a lower concentration than the actual concentration of the component to be detected in the fluid for measurement. Even when fluids for measurement are supplied to the sensor element 10 at the same concentration, the change in the reactive portions 12 may differ slightly. Even if the reactive portions 12 exhibit the same change, the output of the detectors 13 may differ slightly. Accordingly, a reduction in the accuracy of the measurement results from the sensor element 10 is a concern. To address this issue, I performed computer simulations to simulate the concentration calculation of a component to be detected using the above-described principle. I then verified the effect that the selectivity of each reactive portion 12 had on the accuracy of the measurement results.

<Simulations and Observations>

The simulations I performed are described below.

First, the basic simulation method is described. The first step of the simulation was to set the selectivity of the reactive portions 12 (A₁, A₂, B₁, B₂ in Equation 1) to arbitrary fixed values and the concentration of the fluid component for measurement (X_{A}, X_{B} in Equation 1) to arbitrary variables, substitute these into Equation 1, and calculate the sensor output (Y₁, Y₂ in Equations 1, 2). Assuming actual measurement, the sensor output calculated with Equation 1 (Y₁, Y₂) was multiplied by a measurement error. Equation 2 was then determined on the basis of the data set including the concentration (X_{A}) of the fluid component for measurement and the sensor output (Y₁, Y₂). The data set included sufficient data for determining Equation 2.

The second step was to set the selectivity of the reactive portions 12 (A₁, A₂, B₁, B₂) to arbitrary fixed values (the same values as in the first step) and the concentration of the fluid component for measurement (X_{A}, X_{B}) to arbitrary variables (where X_{A} is the same value as in the first step, and X_{B} is a different value from the first step) and substitute these into Equation 1. The sensor output (Y₁, Y₂) of the detectors 13 obtained from Equation 1 was then substituted into Equation 2 to calculate the concentration (X_{A}) of the fluid component for measurement. Assuming actual measurement, the sensor output (Y₁, Y₂) substituted into Equation 2 was multiplied by a different measurement error than in the first step.

In the third step, the difference between the concentration (X_{A}) of the fluid component for measurement set in the second step and the concentration (X_{A}) of the fluid component for measurement calculated in the second step (i.e. the concentration calculation error, described below) was determined.

In the fourth step, the value of the selectivity of the reactive portions 12 (A₁, A₂, B₁, B₂) was changed, and the first through third steps were repeated.

This completes the basic simulation method.

Next, the specific simulations I performed are described.

(First Simulation)

I started by performing a first simulation. In the first simulation, a sensor apparatus 20 having two channels was assumed, and the selectivity of the first channel and the second channel was verified.

In the present disclosure, a channel refers to a combination of a reactive portion and a detector. In other words, one channel is a concept that includes one reactive portion and one detector.

(Setting of Selectivity)

In the first simulation, the ratio of the sensitivity to the component to be detected to the sensitivity to the noise component in the first channel was set to x-to-1, and the value of x was changed over a range from 1 to 30. The ratio of the sensitivity to the component to be detected to the sensitivity to the noise component in the second channel was set to 1-to-y, and the value of y was changed over a range from 1 to 30.

(Setting of Component Concentration in Fluid for Measurement)

In the first simulation, the concentration of each component in the fluid for measurement was set assuming measurement of a slight amount of the component to be detected included in the fluid for measurement. Specifically, the concentration of the component to be detected was changed within a range of 0.1 ppm or more to 10 ppm or less. The concentration of the noise component was set to a random number based on a uniform distribution (a range of 50% to 150%) with 100 ppm as the central value.

(Results and Observations)

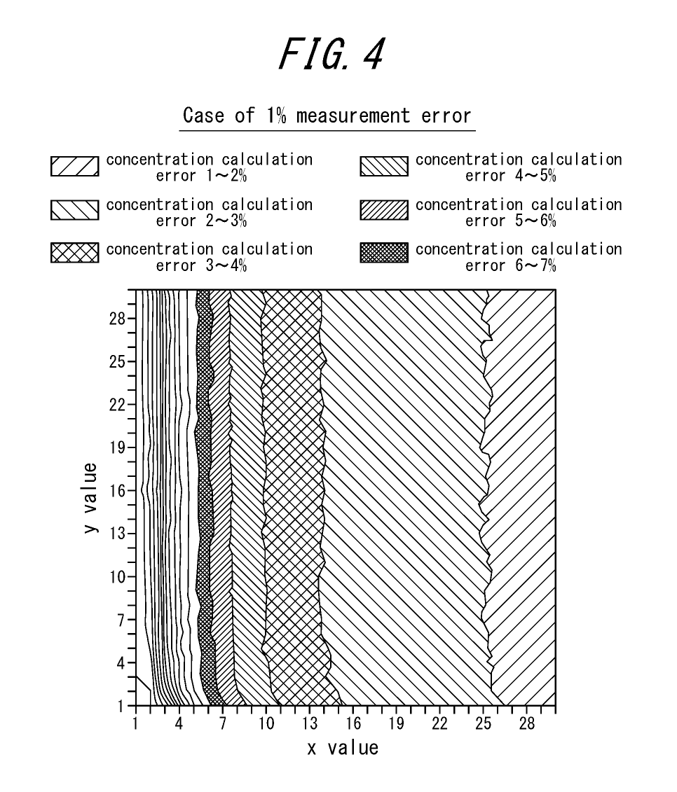

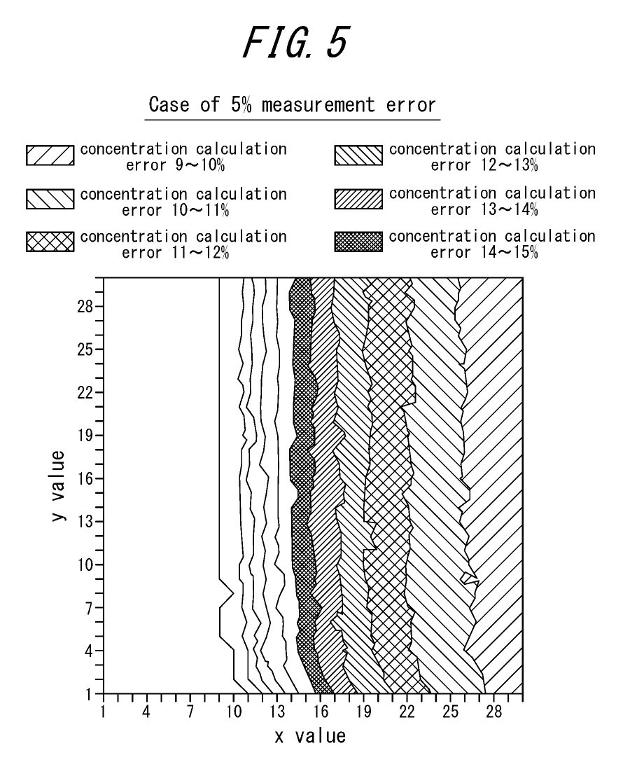

FIGS. 4 and 5 illustrate the results of the first simulation. FIG. 4 illustrates the simulation results for a measurement error of 1%. FIG. 5 illustrates the simulation results for a measurement error of 5%. In FIGS. 4 and 5, the vertical axis represents the value of y, and the horizontal axis represents the value of x. The concentration calculation error in FIGS. 4 and 5 is indicated by different hatching every 1%. In FIG. 4, however, hatching is omitted for regions with a concentration calculation error of 7% or more. Similarly, hatching is omitted in FIG. 5 for regions with a concentration calculation error of 15% or more. The measurement error is a random number based on a normal distribution with the aforementioned numerical value as the central value.

It can be seen from FIGS. 4 and 5 that the concentration calculation error decreases as the value of x is larger. In other words, the accuracy of the measurement result for the component to be detected increases as the selectivity of the first channel with respect to the component to be detected is higher. It can be seen from FIGS. 4 and 5 that if the value of x is constant, the concentration calculation error is nearly constant, regardless of the value of y. In other words, the ratio of the sensitivity to the component to be detected to the sensitivity to the noise component in the second channel has little effect on the accuracy of the measurement result for the component to be detected.

Accordingly, it is clear that an effective way to increase the accuracy of the measurement result for the component to be detected by the sensor apparatus 20 and the sensor element 10 is for one of the reactive portions 12 to have high selectivity to the component to be detected. In other words, if the first reactive portion 12a has higher reactivity than the second reactive portion 12b to the component to be detected in a sample, and the selectivity with respect to the component to be detected is higher than the selectivity with respect to the noise component in the sample other than the component to be detected, then the accuracy of the measurement result for the component to be detected by the sensor apparatus 20 and the sensor element 10 can be improved.

(Second Simulation)

Next, I performed a second simulation. In the second simulation, the selectivity of the second channel was verified for the case of fixing the selectivity of the first channel.

(Setting of Selectivity)

In the second simulation, the ratio of the sensitivity to the component to be detected to the sensitivity to the noise component in the first channel was fixed at 10-to-1. The ratio of the sensitivity to the component to be detected to the sensitivity to the noise component in the second channel was set to z-to-w, and the values of z and w were each changed over a range from 1 to 30.

(Setting of Component Concentration in Fluid for Measurement)

In the second simulation, the concentration of the component to be detected and the concentration of the noise component were set in the same way as in the first simulation.

(Results and Observations)

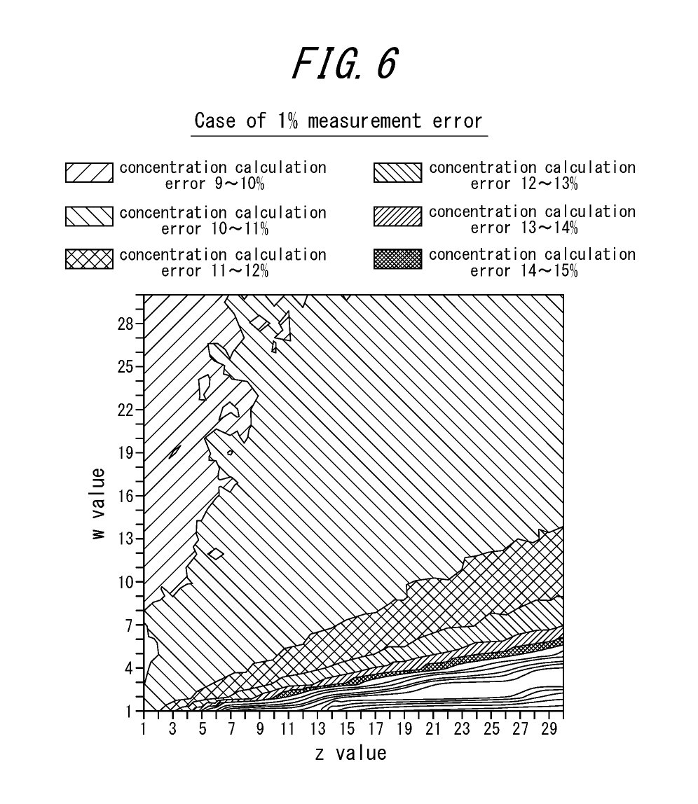

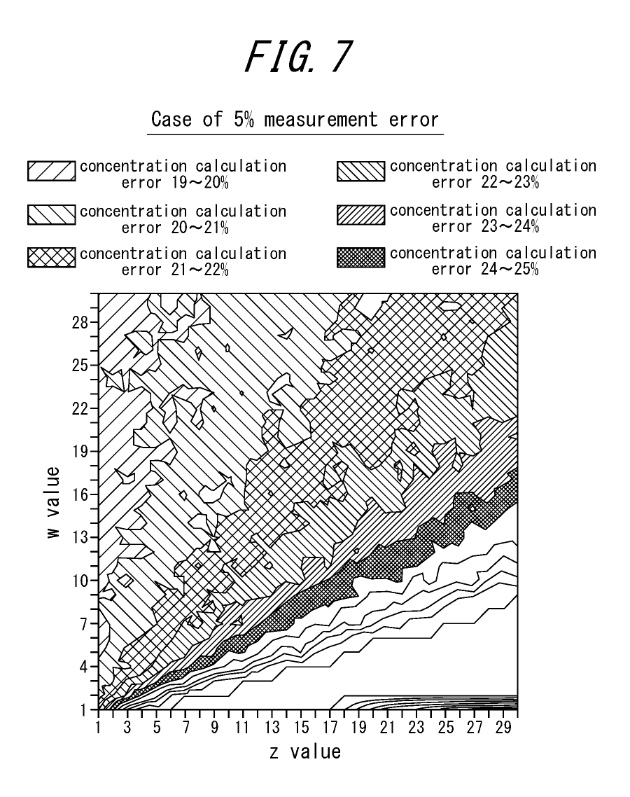

FIGS. 6 and 7 illustrate the results of the second simulation. FIG. 6 illustrates the simulation results for a measurement error of 1%. FIG. 7 illustrates the simulation results for a measurement error of 5%. In FIGS. 6 and 7, the vertical axis represents the value of w, and the horizontal axis represents the value of z. The concentration calculation error in FIGS. 6 and 7 is indicated by different hatching every 1%. In FIG. 6, however, hatching is omitted for regions with a concentration calculation error of 15% or more. Similarly, hatching is omitted in FIG. 7 for regions with a concentration calculation error of 25% or more. The measurement error is a random number based on a normal distribution with the aforementioned numerical value as the central value.

It can be seen from FIGS. 6 and 7 that the concentration calculation error increases as the value of z is larger and as the value of w is smaller. In other words, the accuracy of the measurement result for the component to be detected decreases as the selectivity of the second channel with respect to the noise component is smaller. That is, the greater the selectivity of the second channel with respect to the noise component, the more the accuracy of the measurement result for the component to be detected can be increased.

Accordingly, it is clear that an effective way to increase the accuracy of the measurement result for the component to be detected by the sensor apparatus 20 and the sensor element 10 is for one of the reactive portions 12 to have high selectivity to the noise component. In other words, if the reactivity of the second reactive portion 12b with respect to the component to be detected is lower than the reactivity with respect to the noise component, the accuracy of the measurement result for the component to be detected by the sensor apparatus 20 and the sensor element 10 can be improved.

(Third Simulation)

Next, I performed a third simulation. In the third simulation, the quantity of the channels and the selectivity of each channel were verified, assuming a more realistic measurement.

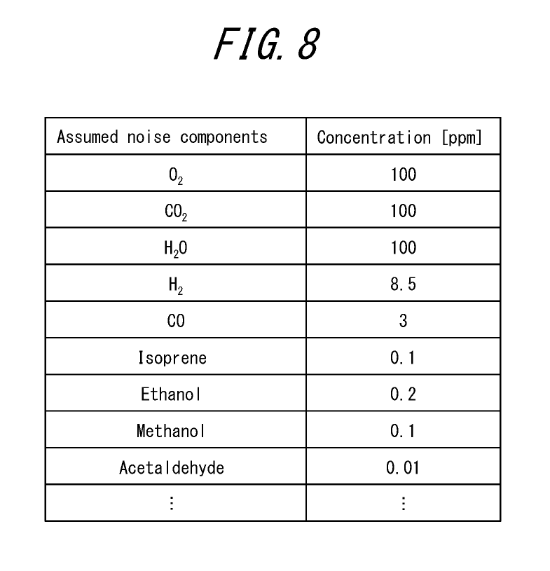

In the third simulation, measurement of human breath was assumed as an example, and the component to be detected was assumed to be acetone. Considering these assumptions, a plurality of types of noise components were also assumed, as illustrated in FIG. 8. In the third simulation, the noise components were classified into two types by concentration: big noise and small noise. The big noise had a higher concentration in the fluid for measurement than the small noise. For example, the big noise may be defined as a gas with a predetermined concentration or higher in the fluid for measurement, and the small noise as a gas with less than the predetermined concentration in the fluid for measurement. Another example is to define the big noise as a gas with a concentration equal to or greater than a predetermined multiple of the maximum concentration of the component to be detected in the fluid for measurement, and the small noise as a gas with a concentration less than the predetermined multiple of the maximum concentration of the component to be detected in the fluid for measurement. In the third simulation, oxygen (O₂), carbon dioxide (CO₂), and water vapor (H₂O) were classified as big noise, and the remaining noise components were classified as small noise.

(Setting of Selectivity)

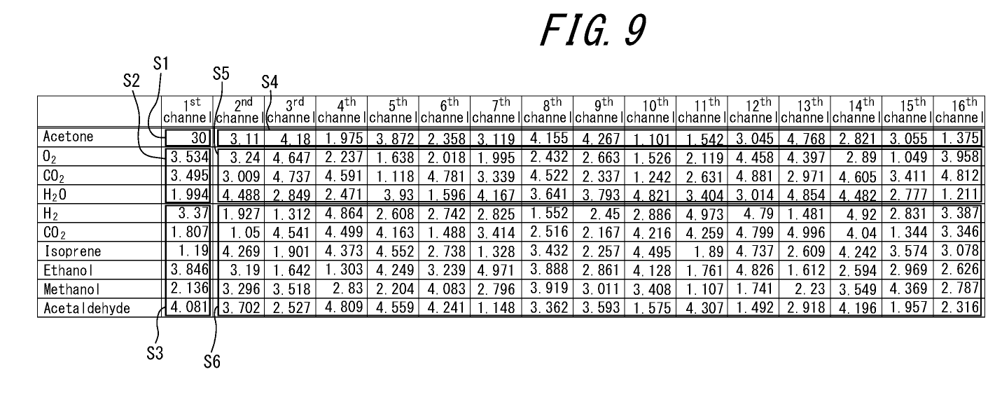

Referring to the results of the first and second simulations, the first channel was set to exhibit the highest selectivity with respect to the component to be detected in the third simulation. In the example illustrated in FIG. 9, the selectivity of the first channel with respect to acetone was set to 30. One of the channels from the second channel onward was set to have higher selectivity with respect to the noise component than to the component to be detected. In the example illustrated in FIG. 9, the selectivity of the second channel with respect to acetone was set to 3.11, and the selectivity with respect to the noise component was set to be equal or higher.

FIG. 9 illustrates an example of the selectivity settings of each channel. The rows in FIG. 9 indicate the components of the fluid for measurement, and the columns indicate the channel numbers. FIG. 9 illustrates an example with 16 channels. The numerical values listed in the table in FIG. 9 indicate the selectivity with respect to each component for each channel. A larger numerical value represents higher selectivity.

In the present disclosure, the selectivity of the first channel with respect to the component to be detected (the selectivity indicated by S1 in FIG. 9) is referred to as “first signal selectivity”. The selectivity of the first channel with respect to big noise (the selectivities indicated by S2 in FIG. 9) is referred to as “first big noise selectivity”. The selectivity of the first channel with respect to small noise (the selectivities indicated by S3 in FIG. 9) is referred to as “first small noise selectivity”. The selectivity of the second channel onward (the second channel through the sixteenth channel in FIG. 9) with respect to the component to be detected (the selectivities indicated by S4 in FIG. 9) is referred to as “second signal selectivity”. The selectivity of the second channel onward with respect to big noise (the selectivities indicated by S5 in FIG. 9) is referred to as “second big noise selectivity”. The selectivity of the second channel onward with respect to small noise (the selectivities indicated by S6 in FIG. 9) is referred to as “second small noise selectivity”.

Specifically, the “first signal selectivity” was set to a predetermined value in the third simulation. A certain selectivity among the “first big selectivity”, “first small noise selectivity”, “second signal selectivity”, “second big noise selectivity”, and “second small noise selectivity” was determined automatically by the computer in a range of 0.000 to 1.000 (0-1). The selectivities other than the aforementioned certain selectivity were determined automatically by the computer in a range of 1.000 to 5.000 (1-5). The third simulation was performed by changing the number of channels in a range of 2 to 16.

(Setting of Component Concentration in Fluid for Measurement)

In the third simulation, measurement of human breath was assumed as described above, and the concentration of the component to be detected was changed within a range of 0.1 ppm or more to 10 ppm or less. A plurality of types of noise components were also assumed, as described above. The concentration of each noise component was set to a random number based on a uniform distribution (a range of 50% to 150%) with the numerical values illustrated in FIG. 8 as the central values.

(Results and Observations)

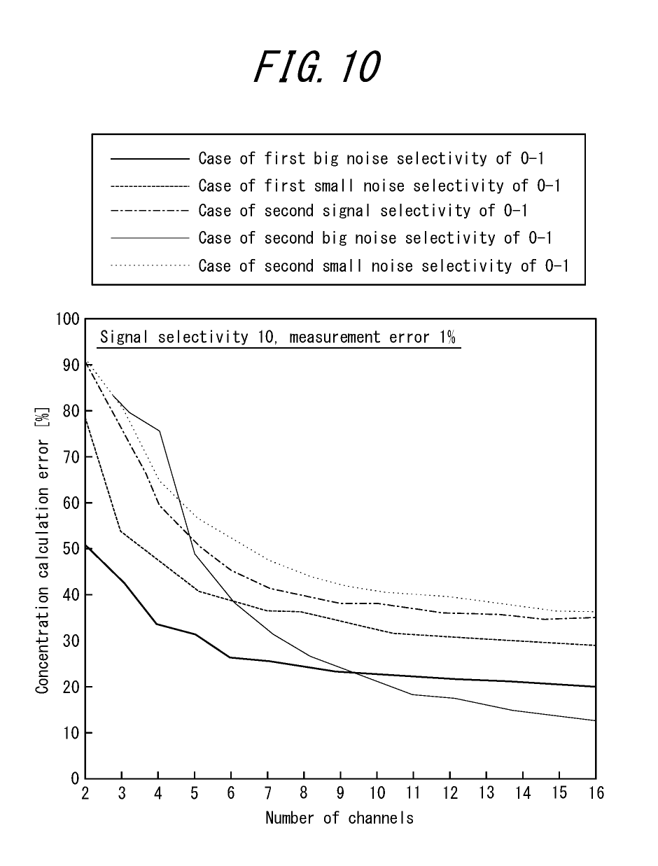

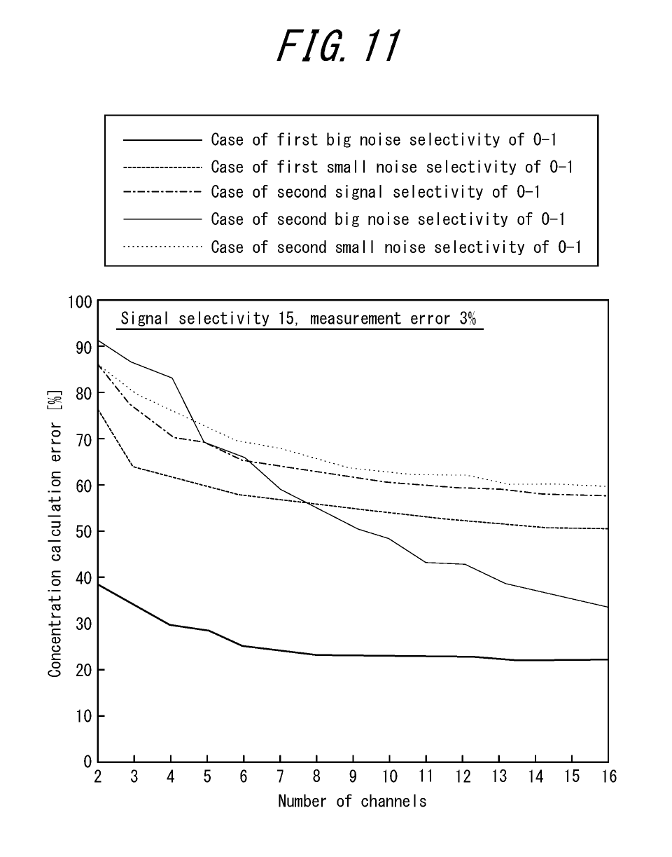

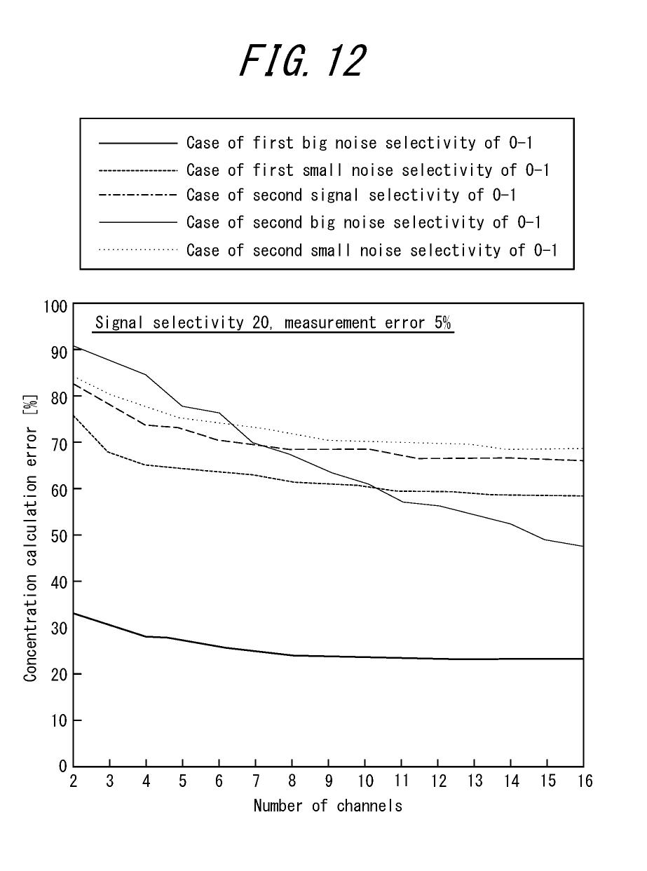

FIGS. 10 to 12 illustrate the results of the third simulation. FIG. 10 illustrates the simulation results for a first signal selectivity of 10 and a measurement error of 1%. FIG. 11 illustrates the simulation results for a first signal selectivity of 15 and a measurement error of 3%. FIG. 12 illustrates the simulation results for a first signal selectivity of 20 and a measurement error of 5%. In FIGS. 10 to 12, the vertical axis represents the concentration calculation error by multiple regression analysis in the simulation, and the horizontal axis represents the number of channels. FIGS. 10 to 12 illustrate the results for each item for which the selectivity range was set to 0-1. The measurement error is a random number based on a normal distribution with the aforementioned numerical value as the central value.

The simulation results in FIGS. 10 to 12 demonstrate that when the first big noise selectivity is in a range of 0-1, the concentration calculation error is smaller than when the other selectivities are in a range of 0-1. In other words, the accuracy of the measurement result for the component to be detected increases when the selectivity of the first channel with respect to big noise is lower than the selectivity of the first channel with respect to small noise. The accuracy of the measurement result for the component to be detected also increases when the selectivity of the first channel with respect to big noise is lower than the selectivity of a channel other than the first channel to the noise component (big noise and small noise).

Accordingly, it is clear that an effective way to improve the accuracy of the measurement result for the component to be detected by the sensor apparatus 20 and the sensor element 10 is for the selectivity with respect to big noise to be lower than the selectivity with respect to small noise in the first channel. In other words, when the noise component is divided into a first noise component and a second noise component contained at a lower concentration than the first noise component, setting the reactivity of the first reactive portion 12a with respect to the first noise component to be lower than the reactivity with respect to the second noise component can improve the accuracy of the measurement result for the component to be detected by the sensor apparatus 20 and the sensor element 10.

It is clear that an effective way to improve the accuracy of the measurement result for the component to be detected by the sensor apparatus 20 and the sensor element 10 is for the selectivity of the first channel with respect to big noise to be lower than the selectivity of a channel other than the first channel to the noise component (big noise and small noise). In other words, if the reactivity of the first reactive portion 12a with respect to the first noise component is lower than the reactivity of a reactive portion other than the first reactive portion 12a, the accuracy of the measurement result for the component to be detected by the sensor apparatus 20 and the sensor element 10 can be improved.

The simulation results in FIGS. 10 to 12 demonstrate that as the number of channels is greater, the accuracy of the measurement result for the component to be detected increases.

The above-described sensor element 10 can be used for various purposes. For example, the sensor element 10 can be used to detect a predetermined gas component in a person's breath. The concentration of the detected gas component can be used to infer the state of the person's body. The inference of the state of a person's body may, for example, refer to the degree of progress of an illness in the body.

The sensor element 10 can, for example, be used to detect a predetermined gas component emitted from a food product. The concentration of the detected gas component can be used to infer the qualities of the food product. The qualities of the food product refer to the properties, quality, or the like of the food product. Examples include the freshness, ripeness, degree of aging, and degree of spoiling of the food product. The sensor element 10 can also be used for various other purposes, such as detecting a predetermined gas component emitted from a device.

Although the present disclosure is based on embodiments and drawings, it is to be noted that various changes and modifications will be apparent to those skilled in the art based on the present disclosure. Therefore, such changes and modifications are to be understood as included within the scope of the present disclosure. For example, the functions and the like included in the various components may be reordered in any logically consistent way. Furthermore, components may be combined into one or divided.

### REFERENCE SIGNS LIST

10 Sensor element
11 Substrate
12 Reactive portion
13 Detector
20 Sensor apparatus
21 Controller
22 Storage

1. A sensor element comprising:
a substrate and a plurality of reactive portions disposed on the substrate and configured to react to a specific component;
wherein the plurality of reactive portions comprise a first reactive portion and a second reactive portion having a lower reactivity than the first reactive portion with respect to a component to be detected in a sample; and
wherein a reactivity of the first reactive portion with respect to the component to be detected is higher than a reactivity of the first reactive portion with respect to a noise component in the sample other than the component to be detected.

2. The sensor element of claim 1, wherein the reactivity of the second reactive portion with respect to the component to be detected is lower than a reactivity of the second reactive portion with respect to the noise component.

3. The sensor element of claim 1,
wherein the noise component comprises a first noise component and a second noise component contained at a lower concentration than the first noise component; and
wherein a reactivity of the first reactive portion with respect to the first noise component is lower than a reactivity of the first reactive portion with respect to the second noise component.

4. The sensor element of claim 3, wherein the reactivity of the first reactive portion with respect to the first noise component is lower than a reactivity of a reactive portion other than the first reactive portion among the plurality of reactive portions.

5. A sensor apparatus comprising:
a sensor element comprising a substrate and a plurality of reactive portions disposed on the substrate and configured to react to a specific component; and
a controller configured to calculate a value related to a component in a sample on the basis of a signal output from the sensor element in accordance with a reaction of the plurality of reactive portions;
wherein the plurality of reactive portions comprise a first reactive portion and a second reactive portion having a lower reactivity than the first reactive portion with respect to the component to be detected; and
wherein a reactivity of the first reactive portion with respect to the component to be detected is higher than a reactivity of the first reactive portion with respect to a noise component in the sample other than the component to be detected.
